(19) United States
(12) Patent Application Publication
(10) Pub. No.: US 2006/0258279 A1
(43) Pub. Date: Nov. 16, 2006

(54) Method for disinfection inside an air lock and method for the implementation thereof

(76) Inventor: Eric Bonnard, GRIVEGNEE (BE)

Correspondence Address: SUGHRUE MION, PLLC, 2100 PENNSYLVANIA AVENUE, N.W., SUITE 800, WASHINGTON, DC, 20037, US

(21) Appl. No.: 10/519,104

(22) PCT Filed: Jun. 26, 2003
(86) PCT No.: PCT/BE03/00110

§ 371 Date: Jan. 20, 2006

(30) Foreign Application Priority Data
Jun. 28, 2002 (BE) 2002/0415

(51) Int. Cl. B05B 15/12

(52) U.S. Cl. 454/55

(57) ABSTRACT

A method for entering into an air lock between an outer volume and an aseptic, pharmaceutical-quality enclosed space, wherein an operator dons a diving-type suit supplied with breathable air, the operator enters the air lock, the suit is disinfected in the air lock by projecting a pulverized sterile disinfectant liquid onto said suit and the disinfectant liquid, which is drained off from the suit and the inner partition walls of the suit during and after disinfecting, is eliminated from the air lock, and turbulent sterile ambient-temperature air currents are blown onto said suit, and the air, which is charged with disinfectant and possibly moisture, is evacuated from the air lock until the suit and the inner partition walls of the air lock are changed, then the operator enters the aseptic space.

The present invention relates to a method of passing into an airlock between an external space and an aseptic enclosure, comprising
the slipping on by an operator of sealed impermeable overalls of the spacesuit type, fed with respirable air,
entry by the operator into the airlock by opening a first access affording passage between the external space and the airlock, in the closed position of a second access allowing passage between the airlock and the aseptic enclosure,
closure of the first access,
disinfection of the overalls in the closed airlock by spraying an atomised disinfectant liquid onto it, and
entry of the operator into the aseptic enclosure by opening the second access, in the closed position of the first access.

It is known that aseptic enclosures, referred to as being of pharmaceutical quality, in which pharmaceutical products are manufactured and/or manipulated, must meet purity criteria which are moreover regulated in known standards (see the Regulations on Medicinal Products in the European Union, Vol 4, Good Manufacturing Practice, Medicinal Products for Human use and Veterinary Medicinal Products, published 1999; 21 Code of Federal Regulations, Parts 210 and 211, US Food and Drug Administration, 29, Sep. 1978).

The major problem posed by such enclosures is that, when the operator enters them, he should not entrain contaminating agents or even simple dust in the enclosure, whether on his clothes or by the contribution of external air which he brings on entering or by breathing. It is of course also important for the operator, where applicable, not to risk being intoxicated by the pharmaceutical substances which he is handling in the enclosure and not to entrain toxic particles with him.

An airlock for passing between the outside and the aseptic chamber has therefore been provided for a long time, at the entry to the these chambers.

In general terms, at the present time, the operator slips on overalls made from synthetic fabric, composed of 3 to 4 pieces, which are sterilised or disinfected in advance and which enable the operator to breathe in the ambient air through a surgical mask and in certain cases to use facial breathing apparatus which sucks in the ambient air and discharges the expired air through a filter. The closed airlock is supplied with sterile air and the airlock is regulated for temperature, pressure and humidity in a manner corresponding to what is found in the aseptic enclosure. Next the operator enters the enclosure.

This method of operating presents several major drawbacks. The operator himself contaminates the outside of the sterilised overalls, not only whilst he is putting them on but also whilst he is wearing them, having regard to the lack of impermeability of his clothing. It has been found that a major proportion of the contaminants present in conventional aseptic enclosures were in fact brought by the operator himself, because of this. In addition, the operator is also obliged, when leaving the enclosure, to remove the overalls and then to slip on new ones if he wishes to enter the enclosure once again.

This gives rise to an enormous cost in maintaining and sterilising or disinfecting the overalls.

A method of passing into an airlock is known, which comprises in particular sterilisation of the overalls within the airlock (see for example U.S. Pat. No. 4,304,224). This method has the drawback that the sterilisation is carried out by the creation of a gaseous atmosphere in which the sterilising agent is atomised and is in a suspension as in a mist. It is difficult under these conditions to design a sterilisation, or even a reproducible disinfecting, effective in the pharmaceutical sense, of the overalls in all its parts, sometimes concealed, such as the underneath the arms, the feet, the crotch, etc. Moreover, the elimination of the disinfecting agent is effected by a complicated process of washing the air in the airlock and heating the washed air, re-introduced into the airlock. Such a heating is in particular to be excluded, since this hot air is favourable to the development of micro-organisms and will be transferred into the enclosure when the access to the aseptic enclosure is opened. These conditions are also difficult for the operator to bear, who is wearing overalls in which the heat of his body remains confined.

An airlock for entering an aseptic enclosure is also known in which the operator wearing overalls is subjected to a shower of disinfectant liquid (see for example U.S. Pat. No. 3,501,213). The passage method used in this case, as described at the start, provides for no step of elimination of the disinfectant before the operator enters the enclosure. Such an airlock would therefore be unacceptable according to the aforementioned pharmaceutical quality standards.

According to FR-2.812.820, finally, provision was made for a multiple airlock and a method of passing between an enclosure containing contaminating materials, toxic or irradiating substances or biologically hazardous media and the external environment.

The major problem therefore consists of leaving such enclosures without any problem for the environment and the constraints are therefore totally different from those with an entry into an enclosure of pharmaceutical quality.

The aim of the present invention is to develop a method of passing into an airlock between an external space and an aseptic enclosure and an airlock which make it possible to comply with the aforementioned pharmaceutical quality standards within the aseptic enclosure, by resolving the problems cited above.

In order to afford a solution to these problems provision has been made, according to the invention, for a method as described at the start, in which the aseptic enclosure is of pharmaceutical quality and the disinfectant sprayed is sterile and in which the method comprises an elimination out of the airlock of the disinfectant liquid which drains from the overalls and internal walls of the airlock during and after the disinfecting, and, after the disinfecting, a blowing into the airlock, onto the overalls, of turbulent streams of sterile air at ambient temperature, after the discharge out of the airlock of the air containing disinfectant and possibly moisture, until drying of the overalls and internal walls of the airlock is obtained. The disinfecting under jets of sprayed sterile disinfectant liquid provides an excellent result on all parts of the overalls. Advantageously moreover, it is possible also to provide a programme of shifting about and movement which the operator must perform during this disinfection so that parts which are difficult for the spraying of liquid to reach are reaching in an assured manner. In addition to the recovery of the disinfectant liquid which drains off, the method provides a blowing of sterile air, preferably at ambient temperature and at a flow greater than 15 m/s, advantageously 20 m/s, onto the overalls, which in this way dry rapidly at the same time as the internal walls of the airlock, whilst a replacement, preferably as complete as possible, of the air containing sterilising agent and possibly moisture with fresh sterile air takes place. Advantageously the pressure inside the enclosure is greater than that of the airlock which, in its turn, is greater than that of the external environment. The airlock is therefore under overpressure compared with the latter. This pressure cascade is in accordance with pharmaceutical standards and protects the atmosphere of the aseptic enclosure. The air which, despite the difference in pressure existing between the enclosure and airlock, enters with it into the aseptic chamber, is at a temperature and relative humidity level identical to those present in the latter.

As a disinfectant liquid, it is possible to use any suitable liquid for this purpose, and in particular a mixture of bleach and soap or products marketed under the name of Dettol or HAC for example.

According to an embodiment of the method according to the invention, it comprises a continuous formation, in the airlock, of a laminar flow of sterile air oriented from top to bottom. As soon as the turbulent air current blowers cease to be in service, there also prevails in the airlock a regulated air conditioning with a laminar flow which constantly directs downwards any particle or droplet in suspension in-the air. Advantageously this air can be recirculated at least partially.

According an improved embodiment of the method according to the invention, it also comprises, in the closed airlock, before disinfection, a prior blowing of turbulent streams of sterile air onto the overalls which detach from the latter particles of the dust type. This pre-blowing thus affords a kind of dry cleaning which de-dusts the overalls. The dust is then immediately entrained downwards and out of the airlock by the laminar flow of the conditioning air.

Embodiments of the method according to the invention are indicated in the accompanying claims.

The invention also concerns an airlock for passing between an external space and an aseptic enclosure, comprising
walls delimiting the airlock, in which there are arranged a first access door allowing, in the open position, passage between the external space and the airlock and a second access door allowing, in the open position, passage between the airlock and the aseptic enclosure,
means of supplying respirable air to sealed impermeable overalls of the spacesuit type worn by an operator present in the airlock, and
means of introducing disinfectant liquid into the airlock.

In an airlock according to the invention, the aseptic enclosure is of pharmaceutical quality and the disinfectant liquid is sterile, the means of introducing sterile disinfectant liquid comprising spray nozzles which are in communication sealingly with a source of disinfectant liquid under pressure and are oriented in the airlock so as to atomise this sterile disinfectant liquid over the whole overalls, and the airlock also comprises means of removing the disinfectant liquid which comprise at least one opening in a bottom part of the airlock which collects the disinfectant liquid which drains off during the spraying, blowing means capable of introducing into the airlock turbulent streams of sterile air at ambient temperature towards the overalls and means of discharging out of the airlock blown air containing disinfectant and possibly moisture.

Other embodiments of the airlock according to the invention are indicated in the accompanying claims.

Other details and particularities of the invention will emerge from the description given below non-limitingly and with reference to the accompanying drawings.

FIG. 1 depicts a view in schematic section through an airlock according to the invention, along the line I-I in FIG. 3.

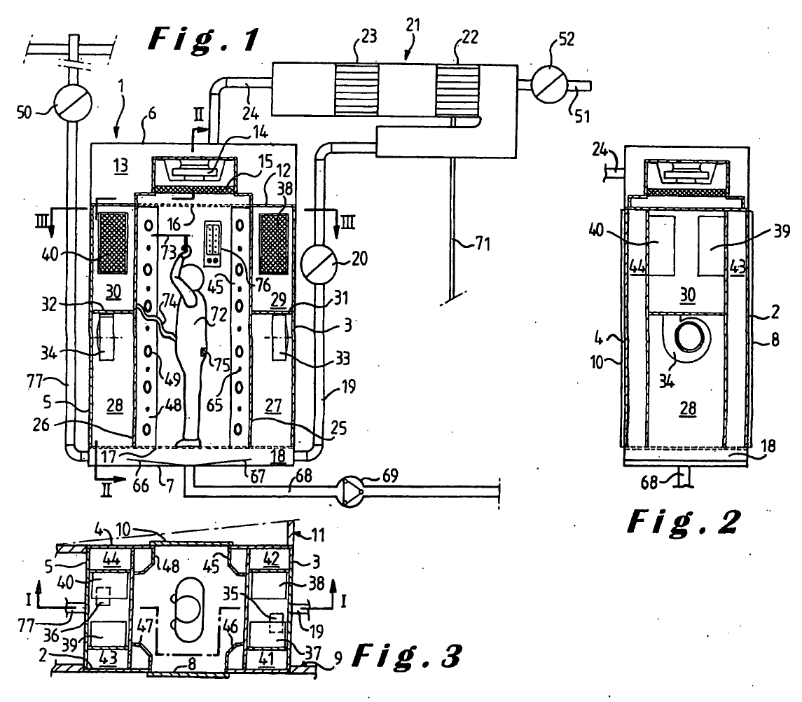

FIG. 2 depicts a view in section along the line II-II in FIG. 1.

FIG. 3 depicts a view in section along the line III-III in FIG. 2.

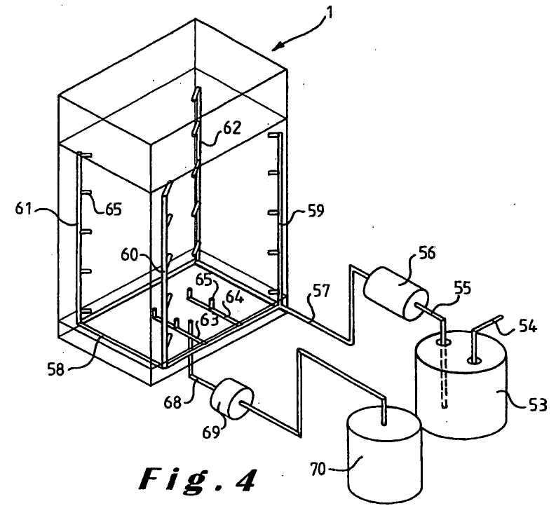

FIG. 4 depicts in a perspective view the disinfectant liquid supply circuit.

In the various figures, the identical or similar elements are designated by the same reference number.

The airlock 1 illustrated in FIGS. 1 to 3 comprises four side walls 2 to 5, a sealing wall 6 and a floor wall 7. Two access doors are arranged in the airlock, the first door 8 allows passage between an external space 9 and the airlock 1 and the second door 10 allows passage between the airlock 1 and the other side of the pharmaceutical-quality aseptic enclosure 11.

In the example embodiment illustrated, there has been provided in the top part of the airlock a false ceiling 12 which allows the formation of a top compartment 13 in which there is mounted a fan 14 which functions continuously and blows an air stream downwards. Below the fan 14 there is arranged a filter 15 of the HEPA type which allows passage of the air blown by the fan 14 in appropriately sterile and dust-free fashion, the air flowing in a laminar flow at the discharge from the filter. Below the filter, in the false ceiling 12, a grille 16 is provided which enables the laminar air stream to flow inside the airlock 1 from top to bottom.

In the bottom part of the airlock according to the example embodiment illustrated, a false floor 17 has been provided in the form of a grille which allows the formation of a bottom compartment 18 in which the air coming from the airlock can escape.

The air collected at the bottom of the airlock in the bottom compartment 18 can, in the example embodiment illustrated, be discharged in a discharge pipe 19, when the recirculation valve 20 is open. The air is then passed into a normal air conditioning unit 21. This unit comprises a condenser 22 which removes from the circuit the excess moisture present in the circulating air, by means of the condensed-liquid pipe 71. The unit 21 also comprises, following the condenser 22, a heating device 23 which heats the air to the ambient temperature required in the airlock. This air, regulated for temperature and humidity level, is then reintroduced into the top compartment 13 of the airlock through the supply pipe 24.

In the example embodiment illustrated, facing the side walls 3 and 5, the airlock has false side walls 25 and 26 which form side compartments each subdivided into two sections, a bottom section 27 and respectively 28 and a top section 29 and respectively 30, by means of partitions 31 and 32. The bottom sections 27 and 28 are in communication with the bottom compartment 18 by means of the grille 17. In each bottom section there is arranged a fan 33 and respectively 34, the discharge 35 and respectively 36 of which opens out in the corresponding top section (see FIG. 3).

In the example embodiment illustrated, in each top 29 and respectively 30 section, there are mounted two filters 38 and 38 and respectively 39 and 40 of the HEPA type, which open out into vertical corner compartments 41, 42 and respectively 43, 44 and which filter the air in an absolutely sterile and dust-free manner. The corner compartments are in communication with air supply sleeves 45, 46, 47 and respectively 48 disposed vertically on the four corners of the internal space of the airlock, that is to say around the operator when the latter is inside the airlock. Ventilation orifices 49 are arranged all along each of the sleeves. When the fans 33 and 34 are put in service, they suck the internal air from the airlock through the bottom, drive it upwards at high speed, through the sterile filters 37 to 40, this sterile air being projected at high speed onto the overalls of the operator.

The air collected at the bottom of the airlock in the bottom compartment 18, for example when the ventilators 33 and 34 are brought into service, can be partially or totally discharged in a discharge pipe 77, when the discharge valve 50 is in the open position. To compensate for this discharge of air, an air inlet pipe 51 provided with an inlet valve 52 is also provided in the recirculation circuit.

For ease of reading the drawings, the disinfectant liquid supply circuit has been shown separately in FIG. 4.

This circuit comprises a reservoir 53 for any suitable disinfectant liquid. This reservoir can be put under pressure by a sterile compressed air source, not shown, which is in communication with the reservoir 53 through the pipe 54. A liquid discharge pipe 55 extends to the bottom of the reservoir 53 and brings the pressurised liquid to a sterilising filter 56. The sterilely filtered disinfectant liquid is transferred to the bottom compartment 18 of the airlock through a transfer pipe 37, where it opens out in a manifold 58. The manifold 58 supplies pressurised sterile disinfectant liquid to the supply pipes 59 to 62 which are housed, in the example embodiment illustrated, inside the vertical sleeves 45 to 48. The manifold can also supply two supply pipes 63 and 64 which are disposed below the grille 17. These supply pipes 59 to 64 are provided over the entire length with spray nozzles 65 directed towards the inside of the airlock. To arrive inside the airlock the nozzles 65 of the pipes 59 to 62 pass through orifices provided for this purpose in the sleeves 45 to 48.

In the compartment 18 there have also advantageously been provided two inclined metal sheets 66 and 67 which promote a flow of the disinfectant liquid, which drains downwards, during the disinfecting, to a disinfectant liquid discharge pipe 68. The liquid is sucked by a pump 69, for example a peristaltic pump, to a residual-liquid reservoir 70.

The operator, in the airlock, wears overalls 72. Overalls must be understood to mean protective clothing, preferably manufactured in a single piece and covering the entire body. This clothing, of the spacesuit type, is impermeable and sealed in both directions.

The operator is supplied with respirable air from an air source, not shown, and this in a known manner not explained in detail, by means of a flexible pipe 74. The air expired by the operator can be discharged through a sterilising filtration cartridge 75, for example of the P3 type according to the aforementioned standards (European Norm EN 12941/2), which is easily replaceable and situated in the bottom of the overalls. Suspension elements 73 can also be provided in the airlock in order to assist the operator to keep his arms raised during the disinfection and drying, and also in order to guarantee the correct positioning of the operator and the reproducibility of the cycle.

The airlock illustrates its functions as follows.

At the start, the valves 50 and 52 are closed and the pump 69, the fans 33 and 34 and the compressed air source from the reservoir 53 are out of service. The recirculation value 20 is in the open position and the fan 14 is in operation. The latter remain in operation throughout the period of use of the airlock 1.

The air conditioning unit regulates the air entering the top compartment 14 at a temperature of 20° C.±2 and at a relative humidity level of 50%±10. The fan 14 affords an airflow speed of 0.45 m/s. The HEPA filter 15 can have for example a surface area of 915 mm×915 mm. The flow rate is set for example at 1350 m³/h. The air thus flows downwards in a linear manner in an internal space of the airlock whose volume is approximately 3.5 m³. The overpressure thus imposed is approximately 30 Pa. This regulation of the airlock allows a renewal of the air contained in the airlock of 200 to 600 time per hour.

The operator slips on working overalls of the spacesuit type, for example in a room of pharmaceutical specification 10.000, these overalls not necessarily having to be sterile. He then enters the airlock, which may comply for example with the standards of a room of pharmaceutical classification 1.0000. To assist him and to force him to follow a complete programme, a control panel 76 is provided inside the airlock. The buttons on the panel each correspond to a distinct command for triggering or stopping a component of the equipment or to a movement of the operator. It is not possible to pass to the following command if the previous one has not been totally accomplished.

After entering the airlock, in the closed position of the door 10, the operator closes the door 8 and places himself in an appropriate position of the feet and hands. For example, electrical sensors are provided on the floor for the feet and, for the hands, the operator must suspend them on the suspension elements 73.

According to one embodiment, the recirculation valve 20 can then be closed, once the vales 50 and 51 are open and the fans 33 and 34 are brought into service. The closure of the valve 20 is however not obligatory. Turbulent air is then blown for approximately 20 seconds at a speed preferably greater than 20 m per second in the direction of the overalls in order to detach the particles of dust adhering to them. This sterile air comes, in the example illustrated, from 24 air ventilation orifices 49.

The valves 50 and 51 are once again closed. The valve 20, if it has been closed, is reset to the open position and the flow of laminar air continues into the airlock, which directs the dust detached from the overalls towards the bottom of the airlock.

The compressed air source of the disinfectant liquid circuit is then started up along with the peristaltic pump 69. The spray nozzles 65 can advantageously by supplied with sterile disinfectant liquid independently of each other. It is possible for example to commence with the nozzles 65 situated below the grille 17. During this spraying, the operator must successively lift his feet in order obtain complete disinfection. Then the 20 spray nozzles 65 disposed in the corners of the airlock are brought into service and disinfect the whole of the overalls under the sprayed disinfectant liquid jets. The residual disinfectant which drains off during this operation is collected downwards through the reservoir 71.

A contact time of approximately for example 5 minutes is observed in order to allow the disinfectant to act. The disinfectant liquid circuit is then switched off, whilst the air conditioning with the laminar air flow is maintained.

After this contact time, the fans 33 and 34 are restarted and the valves 50 and 51 are reset to the open position. A drying step then takes place during which a major part or even all of the air present in the airlock is discharged through the pipe 77 and valve 50, this air being replaced by sterile conditioned air supplied through the pipe 24.

The powerful sterile air jets dry the overalls and the internal walls of the airlock 1. The drying is stopped when the required humidity level is reached in the airlock, and for example after a period of approximately 5 minutes. The pressure inside the airlock has been maintained throughout all the process at approximately 30 Pa. The pressure parameters of the airlock can be modified according to the pressure prevailing in the adjacent aseptic enclosure, so as always to be 5 Pa less than this.

The operator can then enter the aseptic enclosure 11, that is to say for example a room of pharmaceutical classification 100 B/A, by opening the door 10, whilst the door 8 remains closed.

The passage through the airlock has lasted for approximately 15 minutes, including dressing.

If necessary, when leaving the aseptic enclosure, an identical process can be performed in the airlock.

On leaving, the operator can remove his overalls and hang them up whilst waiting until he has to re-enter the enclosure the next time. Intermediate sterilisation of the overalls is no longer necessary.

The invention will be explained in more detail with a help of examples, given non-limitingly,

### EXAMPLE 1

Disinfectant used: Dettol 5% in mains H₂O

De-dusting phase: 20 seconds

Disinfection phase:

Spraying: 40 seconds

Total contact time: 3 minutes

Quantity of disinfectant used: 6.5 litres

Drying phase: 6 minutes

Verification of drying by visual examination: OK

Microbiological sampling: see table below

### EXAMPLE 2

Disinfectant used: Dettol 5% in mains H₂O

De-dusting phase: 20 seconds

Disinfection phase:

Spraying: 100 seconds

Total contact time: 3 minutes

Quantity of disinfectant used: 6.5 litres

Drying phase: 5 minutes

Verification of drying by visual examination: OK

Microbiological sampling: see table below

### EXAMPLE 3

Disinfectant used: Dettol 5% in mains H₂O

De-dusting phase: 20 seconds

Disinfection phase:

Spraying: 20 seconds

Total contact time: 5 minutes

Quantity of disinfectant used: 3.25 litres

Drying phase: 5 minutes

Verification of drying by visual examination: OK

Microbiological sampling: see table below

### EXAMPLE 4

Disinfectant used: HAC 1% in mains H₂O

De-dusting phase: 30 seconds

Disinfection phase:

Spraying: 50 seconds

Total contact time: 5 minutes

Quantity of disinfectant used: 6.5 litres

Drying phase: 5 minutes

Verification of drying by visual examination: OK

Microbiological sampling: see table below

### EXAMPLE 5

Disinfectant used: Dettol 5% in demineralised H₂O

De-dusting phase: 20 seconds

Disinfection phase:

Spraying: 100 seconds

Total contact time: 5 minutes

Quantity of disinfectant used: 8.5 litres

Drying phase: 5 minutes

Verification of drying by visual examination: OK

Microbiological sampling: see table below

### EXAMPLE 6

Disinfectant used: Dettol 5% in demineralised H₂O

De-dusting phase: 20 seconds

Disinfection phase:

Spraying: 90 seconds

Total contact time: 5 minutes

Quantity of disinfectant used: 5.25 litres

Drying phase: 5 minutes

Verification of drying by visual examination: OK

Microbiological sampling: see table below

TABLE
Microbiological samples taken on the overalls, by culture medium (Contact boxes)
Standard: results <5 cfu (colony formation unit) per sample

| | Example 1 | | Example 2 | | Example 3 | | Example 4 | | Example 5 | | Example 6 | |
|---|---|---|---|---|---|---|---|---|---|---|---|---|
| | A | B | A | B | A | B | A | B | A | B | A | B |
| Sampling site | cfu | cfu | cfu | cfu | cfu | cfu | cfu | cfu | cfu | cfu | cfu | Cfu |
| Right forearm | 0 | 3 | 0 | 0 | 2 | 2 | 0 | 0 | 0 | 0 | 1 | 1 |
| Left forearm | 0 | 0 | 0 | 0 | 1 | 1 | 0 | 0 | 0 | 0 | 0 | 0 |
| Right chest | 1 | 1 | 0 | 0 | 0 | 0 | 0 | 0 | 0 | 0 | 0 | 0 |
| Left chest | 1 | 1 | 0 | 0 | 0 | 0 | 1 | 2 | 0 | 0 | 1 | 1 |
| Right thigh | 0 | 1 | 0 | 0 | 0 | 1 | 0 | 0 | 0 | 0 | 0 | 0 |
| Left thigh | 0 | 0 | 0 | 0 | 2 | 3 | 0 | 0 | 0 | 0 | 0 | 0 |
| Right ankle | 0 | 0 | 0 | 0 | 2 | 2 | 0 | 0 | 0 | 0 | 0 | 1 |
| Left ankle | 0 | 0 | 0 | 0 | 0 | 0 | 1 | 1 | 0 | 0 | 0 | 0 |
| Crotch | 0 | 0 | 0 | 0 | 0 | 0 | 0 | 0 | 0 | 0 | 0 | 0 |
| Head | 0 | 0 | 0 | 0 | 0 | 1 | 0 | 0 | 0 | 0 | 0 | 0 |

A = reading after two days incubation at 37° C.
B = reading after 5 days additional incubation at 20° C.-25° C.

This table shows that all the tests are in accordance with required standards.

It must be understood that the present invention is in no way limited to the embodiments described above and that many modifications can be made thereto without departing from the scope of the accompanying claims.

It would be possible for example to provide a turbulent sterile air source situated outside the airlock in order to supply the fans 33 and 34.

It would also be possible to imagine, after the disinfection step, a step of rinsing off the disinfectant by means of sterile water. In this case, a water pipe can be connected by means of a valve, just upstream of the filter 56.

Before entering the aseptic enclosure, the operator can also put on a pair sterile gloves, as an additional precaution.

1. Method of passing into an airlock between an external space and an aseptic enclosure, comprising
the slipping on by an operator of sealed impermeable overalls of the spacesuit type, fed with respirable air,
entry by the operator into the airlock by opening a first access affording passage between the external space and the airlock, in the closed position of a second access allowing passage between the airlock and the aseptic enclosure,
closure of the first access,
disinfection of the overalls in the closed airlock by spraying an atomised disinfectant liquid onto it, and
entry of the operator into the aseptic enclosure by opening the second access, in the closed position of the first access,
characterised in that the aseptic enclosure is of pharmaceutical quality and the disinfectant sprayed is sterile and in that the method comprises an elimination out of the airlock of the disinfectant liquid which drains from the overalls and internal walls of the airlock during and after the disinfecting, and, after the disinfecting, a blowing into the airlock, onto the overalls, of turbulent streams of sterile air at ambient temperature, after the discharge out of the airlock of the air containing disinfectant and possibly moisture, until drying of the overalls and internal walls of the airlock is obtained.

2. Method according to claim 1, characterised in that it comprises a continuous formation, in the closed airlock, of a laminar flow of sterile air oriented from top to bottom.

3. Method according to one of claims 1, characterised in that it also comprises, in the closed airlock, before disinfection, a prior blowing of turbulent streams of sterile air onto the overalls, in order to detach particles of the dust type therefrom.

4. Method according to claim 1, characterised in that it comprises a supply of respirable air to the overalls from a source of compressed air in sealed communication with the overalls through a flexible pipe, and a discharge of the respirated air out of the overalls with filtration in a sterile manner of this respirated air through a filter situated in a bottom part of the overalls.

5. Method according to claim 1, characterised in that it comprises a programme of movement of the operator allowing disinfection by the disinfectant liquid and drying thereof on all parts of the overalls.

6. Method according to claim 1, characterised in that the turbulent streams of sterile air are blown into the airlock at a speed of flow of at least 15 m/s, preferably at least 20 m/s.

7. Method according to claim 1, characterised in that the aseptic enclosure has a first pressure, the airlock has a second pressure less than the first and the external space has a third pressure less than the second.

8. Airlock (1) for passing between an external space (9) and an aseptic enclosure (11), comprising
walls (2-7) delimiting the airlock, in which there are arranged a first access door (8) allowing, in the open position, passage between the external space and the airlock and a second access door (10) allowing, in the open position, passage between the airlock (1) and the aseptic enclosure (11),
means of supplying respirable air (74) to sealed impermeable overalls (72) of the spacesuit type worn by an operator present in the airlock, and
means of introducing disinfectant liquid into the airlock,
characterised in that the aseptic enclosure is of pharmaceutical quality and the disinfectant liquid is sterile, the means of introducing sterile disinfectant liquid comprising spray nozzles (65) which are in communication sealingly with a source of disinfectant liquid under pressure (53) and are oriented in the airlock so as to atomise this sterile disinfectant liquid over the whole overalls, and in that the airlock also comprises means of removing the disinfectant liquid which comprise at least one opening (17) in a bottom part of the airlock which collects the disinfectant liquid which drains off during the spraying, blowing means (49) capable of introducing into the airlock turbulent streams of sterile air at ambient temperature towards the overalls and means (50, 77) of discharging out of the airlock blown air containing disinfectant and possibly moisture.

9. Airlock according to claim 8, characterised in that the spray nozzles (65) are disposed along pipes supplying disinfectant liquid (59 to 64), arranged vertically around the operator inside the airlock and possibly horizontally below him, these supply pipes being connected to the said source of disinfectant liquid (53).

10. Airlock according to claim 9, characterised in that the blowing means comprise ventilation orifices (49) which are disposed along air supply sleeves (48) arranged vertically around the operator inside the airlock, are oriented towards them and are supplied by at least one pressurised air source (33, 34) and in that the means of discharging the laden air comprise at least one outlet opening (17) disposed in a bottom part of the airlock, which is in sealed communication with a discharge pipe (77).

11. Airlock according to claim 8, characterised in that it comprises means of forming a laminar flow of sterile air, (14, 15) between an air inlet situated at the top of the airlock and air outlet situated at the bottom of it and possibly means for at least partial recirculation of the air (19-24) from the said air outlet towards the said air inlet.

12. Airlock according to claim 8, characterised in that the overalls (72) comprise, in a bottom part, a filter (75) allowing discharge with sterile filtration of the air fed into and sucked out of the overalls.
